(19) United States
(12) Patent Application Publication
(10) Pub. No.: US 2013/0006112 A1
(43) Pub. Date: Jan. 3, 2013

(54) Apparatus and method for non-invasively locating blood vessels

(76) Inventor: Terence Vardy, Tweed Heads (AU)

(21) Appl. No.: 13/520,655

(22) PCT Filed: Jan. 6, 2011
(86) PCT No.: PCT/AU2011/000011

§ 371 Date: Sep. 20, 2012

(30) Foreign Application Priority Data
Jan. 6, 2010 (AU) 2010900047

(51) Int. Cl. A61B 8/13; A61B 6/00; A61B 8/00

(52) U.S. Cl. 600/443; 600/437

(57) ABSTRACT

An apparatus (10) for non-invasively location of blood vessels within a portion of body, such as an arm (20) is provided. The apparatus (10) contains an infrared portion and an ultrasound portion, preferably integrated therein. In use, the apparatus (10) is placed above a portion of a body to be scanned such as an arm (20), infrared radiation and ultrasonic energy from the relevant infrared portion and ultrasonic portion is then directed onto the body portion. The reflected infrared radiation is then measured to determine the location of a first vessel type while the reflected ultrasonic energy is utilised to determine the location of a second vessel type within the body portion being scanned.

### BACKGROUND OF THE INVENTION

1. Field of the Invention

The invention relates to an apparatus and method for non-invasively locating blood vessels and, more particularly, to non-invasively locating blood vessels in a portion of a body utilising a combination of infra-red and ultrasound.

2. Discussion of the Background Art

In many medical procedures it is necessary to locate a vessel, such as a vein or artery, in a portion of a body. Typically the portion of the body is an arm, and the procedure involves locating a suitable vessel for placement of a needle to for either an injection or blood collection.

Locating the vessel can be difficult, and in some cases multiple penetrations are necessary to correctly locate the needle in an appropriate vessel. In an effort to improve the visibility of veins, an imaging system is disclosed in U.S. Pat. No. 6,556,858, the entire disclosure of which is incorporated herein by reference. The imaging system utilises infrared light to locate veins, and projects an enhanced contrast image of the veins on the patient's skin. This enables the medical practitioner to more readily locate veins, particularly if they're located under significant deposits of subcutaneous fat. However, the projection of the enhanced contrast image is not as effective on darker skin tones and in bright sunlight. Furthermore, arterial vessels are not determined or distinguished.

A method and apparatus for measuring blood flow in vessels, such as arterial vessels, is disclosed in U.S. Pat. No. 4,103,679, the entire disclosure of which is incorporated herein by reference. The method/apparatus utilise ultrasonic energy bursts from a transducer array and Doppler shifts to determine the blood flow in the vessels.

However, venous vessels are not able to be determined due at least partially to different haemoglobin characteristics in the arterial and venous vessels.

A further disadvantage of both systems is that other objects, such as needles, are not detected, and locating of the object relative to the vessels can only be determined visually on the portion of the body being scanned. This can lead to alignment issues, for example between the needle tip and the actual location of a target vessel.

Clearly it would be advantageous to provide to provide an apparatus and method for non-invasively locating blood vessels which ameliorates at least some of the aforementioned disadvantages of the prior art systems discussed above, or which at least provides a useful alternative.

### SUMMARY OF THE INVENTION

### Disclosure of the Invention

Accordingly in one aspect of the present invention, there is provided an apparatus for non-invasively locating blood vessels within a portion of a body, the apparatus comprising:
a portion utilising infrared to locate at least some of the vessels, and
a portion utilising ultrasound to locate at least some of the vessels.

In yet another aspect of the present invention there is provided an apparatus for non-invasively locating vessels within a region of interest within a body, the apparatus comprising:
at least one infrared source for directing infrared radiation toward the region of interest;
at least one infrared sensor for receiving infrared radiation reflected from said region of interest;
at least one ultrasonic transducer for directing ultrasonic energy toward the region of interest;
at least one acoustic senor for receiving ultrasonic energy reflected from said region of interest; and
wherein the apparatus is adapted to locate a first vessel type within the area of interest based on the reflected infrared radiation and to locate a second vessel type within the area of interest based on the reflected ultrasonic energy.

In yet another aspect of the present invention there is provided a system for non-invasively locating vessels within a region of interest within a body, said system comprising:
an apparatus, said apparatus including:
   at least one infrared source for directing infrared radiation toward the region of interest;
   at least one infrared sensor for receiving infrared radiation reflected from said region of interest;
   at least one ultrasonic transducer for directing ultrasonic energy toward the region of interest;
   at least one acoustic senor for receiving ultrasonic energy reflected from said region of interest;
at least one processor coupled to said apparatus wherein said at least one processor is adapted to:
   determine the locations of vessels of a first vessel type within the area of interest based on the reflected infrared radiation; and
   determine the locations of vessels of a second vessel type within the area of interest based on the on the reflected ultrasonic energy.

Preferably the portion utilising infrared primarily locates venous vessels, and the portion utilising ultrasound primarily locates arterial vessels. Without wishing to be limited by theory, the different methods of detecting the position of veins and arteries is used by the inventor mainly due to the difference in the absorptive spectra of veins and arteries. Typically veins are at a lower temperature than arteries and therefore, will normally absorb a higher level of infrared spectra when compared to arteries which are often at a temperature which is very similar to that of the surrounding tissues rendering an infrared location mechanism for arterial vessels less reliable.

Preferably the apparatus further comprises an output means. The output means may be a data output means for transferring data relating to the location of the vessels. The output means may comprise one or more cables for digital and/or analogue communication of the data. The output means may also comprise a transmitter for wireless transmission of the data (e.g. by Bluetooth, or over a wireless network). Preferably the output means comprises a display.

The display preferably illustrates the located vessels thereon (e.g. by generating an image of the portion of the body with the vessels). Preferably the venous vessels and arterial vessels are displayed simultaneously, but may also be distinguishable (e.g. illustrated in different colours, switchable between each, or the like). The display may be any suitable display means including, for example, an LCD, CRT, TV, projector, or a laptop or computer display. Preferably the display is portable display, such as a handheld display. Even more preferably, the display is a hand held touch screen display. The apparatus may comprise at least one input means and, in a preferred embodiment, the touch screen is a primary input means.

Preferably the portion utilising infrared comprises an infrared light source which is preferably omnidirectional, provided from a plurality of directions, and/or diffused. In use, infrared light reflected from the portion of the body is preferably detected by at least one infrared sensing element. The infrared detector detects infrared radiation reflected from the patient's tissue. In contrast, infrared radiation is absorbed rather than reflected by the patient's veins. As the device is passed over a patient's skin, infrared light from the infrared light source penetrates through the patient's skin and is absorbed by veins under the skin but scattered by the fat and other tissues surrounding the veins allowing identification of the position of veins.

The information to be displayed on the display may be transferred wirelessly or via a cable. Additional information may also be displayed such as, for example, information relating to the vessels and/or the portion of the body may be overlaid, or may at least be accessible (e.g. via a menu or button).

Data relating to the located vessels is preferably captured by the apparatus. The captured data is preferably stored, locally on the apparatus and/or remotely. Preferably the data is at least stored remotely in a database by the apparatus. Even more preferably the data is stored in at least one database entry relating to a person or entity that the portion of the body belongs to. The data may be compared with previously captured data to identify and/or illustrate changes and/or trends in the vessels of the portion of the body.

Preferably the portion utilising ultrasound measures blood flow of located vessels. The vessels may be located utilising ultrasound by measuring the blood flow of at least some of the vessels in the portion of the body, and thereby locating the vessels by noting the measured blood flow located therein. Preferably the ultrasound portion comprises at least one ultrasonic transducer. Preferably the ultrasound portion comprises an array of ultrasonic transducers. In use, preferably the ultrasound portion transmits at least one beam of pulsed ultrasonic energy in the direction of the portion of the body. Preferably a plurality of beams is transmitted towards the portion of the body and, even more preferably, at different angles relative to the portion of the body. Preferably the ultrasound portion comprises at least one receiver for receiving reflected ultrasonic energy.

The direction of blood flow may be determined utilising Doppler frequency shift detection. Range gating techniques may also be applied to ascertain the blood velocity relationship to the cross-sectional dimensions of a vessel.

Preferably the apparatus can detect at least certain objects, such as a needle. The apparatus may detect the needle utilising the infrared portion and/or ultrasound portion, or by utilising a dedicated object locating means. For example, a dedicated object locating means may comprise a magnetised array that detects and locates a magnetised needle tip position relative to the vessels and/or portion of the body.

Preferably the location of the needle can be determined utilising at least the ultrasound portion of the apparatus. Even more preferably, the needle location can be determined in three dimensions within the portion of the body.

Preferably the portion utilising infrared and the portion utilising ultrasound are at least partially comprised on a portable scanner that is hand held and may be manoeuvred over the portion of the body. The portable scanner may be wireless or may be wired to another portion such as, for example, a base station or the display.

In use, a portion of the portable scanner may require direct contact with the surface of the portion of the body, or at least have a sensor placed on the portion of the body. The portion of the portable scanner which may require direct contact is preferably related to the ultrasound portion of the apparatus. However, in a preferred form the portable scanner can be utilised from a distance (i.e. no contact with the portion of the body is required).

According to a another aspect of the present invention, there is provided a method of non-invasively locating blood-vessels within a portion of a body; the method comprising the steps of:
locating at least some of the vessels using infrared; and
locating at least some of the vessels using ultrasound.

Preferably the step of locating at least some of the vessels using infrared comprises emitting infrared light and measuring reflected infrared light from the portion of the body. Preferably the step of locating at least some of the vessels using ultrasound comprises emitting ultrasonic energy and measuring reflected ultrasonic energy from the portion of the body. Preferably the vessels located using infrared are venous, and the vessels located using ultrasound are arterial.

Preferably the method further comprises the step of outputting data relating to the vessels. In a preferred embodiment, the data is output to a display, such as an LCD display. Preferably the data is presented visually (e.g. as an image, graphic, or video) on a display, instead of being only output as raw data. However, raw data is preferably also output and may be received, stored, and/or processed, preferably by a computer. The method may comprise the step of receiving input from an operator and/or computer. Preferably the operator inputs instructions utilising a touch screen display.

The method may further comprise the step of locating a portion of an object, such as a needle, relative to the vessels. This step may also comprise identifying a particular location to place the object. For example, if the object is a needle an ideal location to place the needle may be identified. The identification may be performed by a calculation unit (e.g. by a computer, microprocessor/microcontroller, or the like) or manually (e.g. by visual inspection of output data by an operator).

Preferably the method further comprises the step(s) of storing, retrieving, and/or comparing data in a database. Preferably the database has an entry for the person or entity that the portion of the body belongs to. The method may further comprise the steps of checking to see if a user has a file; if not, opening a file; and storing output data in the file.

The method may further comprise the step of utilising the vessel information and/or output data as a biological identification means. For example, unique vessel information of a portion of a person, such as their hand, may be stored and then utilised for identification purposes.

Throughout the specification it is to be understood that the term infrared is utilised to refer to electromagnetic radiation occurring in the infrared bands of the electromagnetic spectrum and is intended to include near-infrared, short-wave infrared, mid-wave infrared, long-wave infrared and far-infrared bands.

Throughout the specification, including the claims, where the context permits, the term “comprise” and variants thereof such as “comprises” or “comprising” are to be interpreted as including the stated integer or integers without necessarily excluding any other integers.

### BRIEF DETAILS OF THE DRAWINGS

In order that this invention may be more readily understood and put into practical effect, reference will now be made to the accompanying drawings, which illustrate preferred embodiments of the invention, and wherein:

FIG. 1 illustrates a portion of an embodiment of the invention in use over an arm;

FIG. 2 illustrates a display according to an embodiment of the invention;

FIG. 3 is a flow chart illustrating a portion of a vessel viewing system;

FIG. 4 is a flow chart illustrating a portion of a vessel viewing system;

FIG. 5 is a flow chart illustrating a portion of a vessel viewing system;

FIG. 6 is a flow chart illustrating a portion of a vessel viewing system;

FIG. 7 is a flow chart illustrating a portion of a vessel viewing system;

FIG. 8 is a flow chart illustrating a portion of a vessel viewing system;

FIG. 9 is a flow chart illustrating a portion of a vessel viewing system;

FIG. 10 is a flow chart illustrating a portion of a vessel viewing system;

FIG. 11 is a flow chart illustrating a portion of a vessel viewing system;

FIG. 12 is a flow chart illustrating a portion of a vessel viewing system; and

FIG. 13 is a perspective view of an embodiment of the invention.

### DESCRIPTION OF EMBODIMENTS OF THE INVENTION

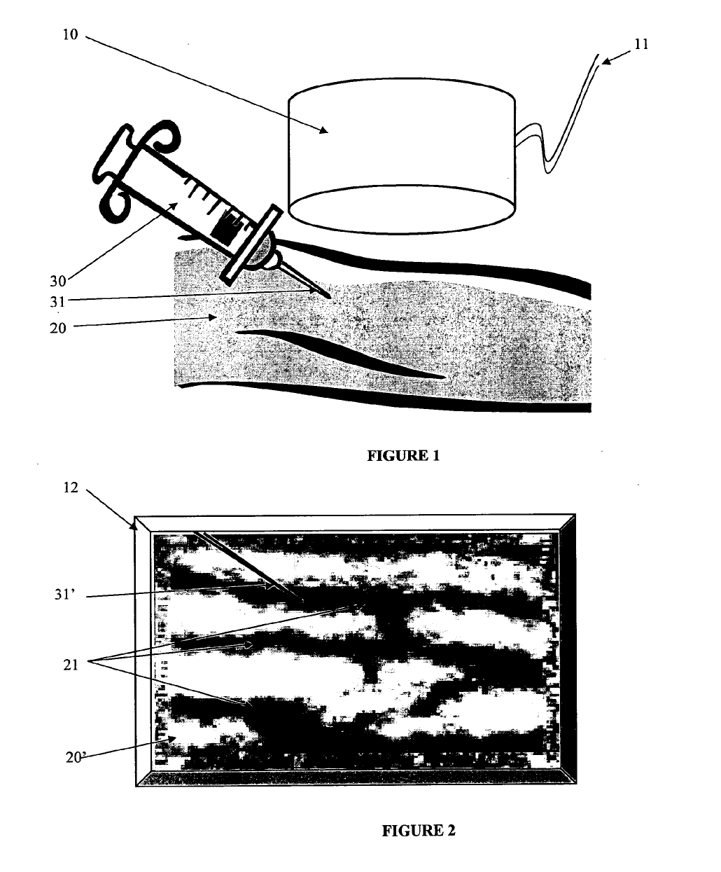

FIG. 1 illustrates a handheld portion of an apparatus 10 that non-invasively locates blood vessels within a portion of body, such as an arm 20. The apparatus 10 contains an infrared portion and an ultrasound portion, preferably integrated therein. In use, the apparatus 10 is placed above a portion of a body to be scanned such as an arm 20 (as illustrated in FIG. 1). The apparatus 10 has a cable 11 for communicating with a base station and/or a display 12 (illustrated in FIG. 2). Although the apparatus 10 is illustrated with a cable 11, it will be appreciated that data may be communicated wirelessly.

Also illustrated in FIG. 1 is a syringe 30 having a needle 31. In traditional use, the syringe operator visually inspects the arm and visually locates an appropriate blood vessel to penetrate with the needle 31. Unfortunately this method of locating a vessel is relatively inaccurate, and multiple penetrations of an arm 20 may be required if an appropriate vessel is missed.

FIG. 2 illustrates a display 12 according to an aspect of the invention. The display 12 illustrates the portion of the arm 20 being scanned by the apparatus 10 as indicated generally by 20′. The infrared and ultrasound scanning of the portion of the arm 20 results in a clear distinction of the different types of vessels (namely veins and arteries) as indicated generally by 21. Although not shown clearly in FIG. 2, preferably the veins and arteries are displayed simultaneously on the display, and even more preferably the veins and arteries are distinguishable from each other (e.g. by being displayed in different colours).

The needle 30 is also depicted on the display 12 as 30′. The operator of the needle 30 is thereby able to accurate locate a suitable vein or artery to penetrate with the needle 30. Preferably the display us updated in real time, so the operator has a negligible delay between their movements of the needle 30 and the depiction of the location of the needle 30′ being moved relative to the arm 20′.

The display 12 is preferably a touch screen display that accepts input from an operator. Such input may include, for example, changing settings of the apparatus (e.g. brightness, contrast, audible notifications, calibration etc.), changing the information and/or data displayed, and turning the device on and off. Alternative (or additional) input means may also be provided in the form of, for example, a keyboard, receiving data from a computer or other device, a mouse or other pointing device, and the like.

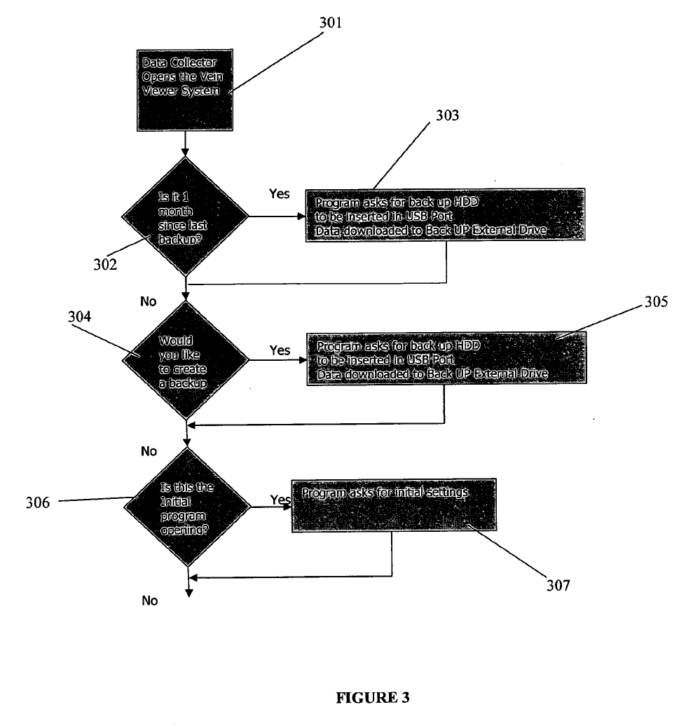

FIG. 3 depicts a flow chart illustrating the initialisation procedure for a vessel viewing system according to one embodiment of the invention. As shown on commencement of the initialisation procedure 301 the system determines whether a backup of all system data is necessary 302. In the depicted example the backup is prompted based on the amount of time between successive backups. If the time between backups exceeds a preset threshold (in this case the threshold is set to 1 month between successive backups) the system commences a backup by prompting the user to specify a destination such as a drive (e.g. an external hard drive or other removable media e.g. a flash or USB drive or optical storage media such as a CD/DVD) or directory to which the backup is to be stored 303 before proceeding to create the backup data file.

If the length of time between backups does not exceed the threshold value then the system prompts the as to whether they wish to perform a backup 304. If the user elects to make a backup, the system then request the user to specify a destination drive (e.g. an external hard drive or removable media g. a flash or USB drive or optical storage media such as a DVD) or directory to which the backup is to be stored 305. Once the user has decided whether or not to create a backup 304 the system then determines whether this is the first time the system has commenced its operation 306, if so, the system proceeds to prompt the user to enter initial system settings 307.

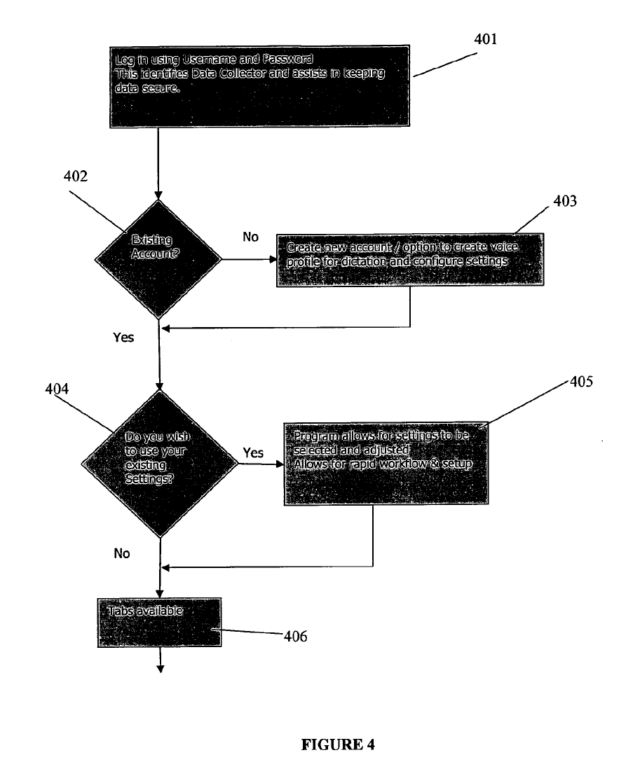

Once the initialisation procedure is complete, the system proceeds to the account creation verification phase per the flow chart illustrated in FIG. 4. As shown the system prompts the user for their credentials to effect system login 401 e.g. user name and password. Alternatively, other forms of user identification such as, for example, biometric identification i.e. finger print scan, rental scan, voice print verification or combination thereof could be utilised to effect system login. Once logged in the system then determines whether the user has an existing account 402 on the system. If so the system then presents the user with the option to change their existing setting 404. Should the user elect to modify their existing settings they are then presented with various setting selections 405.

In event that the user does not have an existing account the system proceeds to create an account for the user 403. During account creation the user has the option to create a voice profile for various functions such as dictation and voice print ID where biometric identification is provided. In addition to the creation of the voice profile the user may also configure a number of personal settings relating to the system. Once the account setup is complete the system then proceeds to step 404 discussed above. It will of course be appreciated by those of skill in the art that for newly created accounts the option to alter existing settings is in all likelihood to be answered in the negative, unless of course the user has exited account set up prematurely in which case they can adjust their setting by entering step 405 as discussed above.

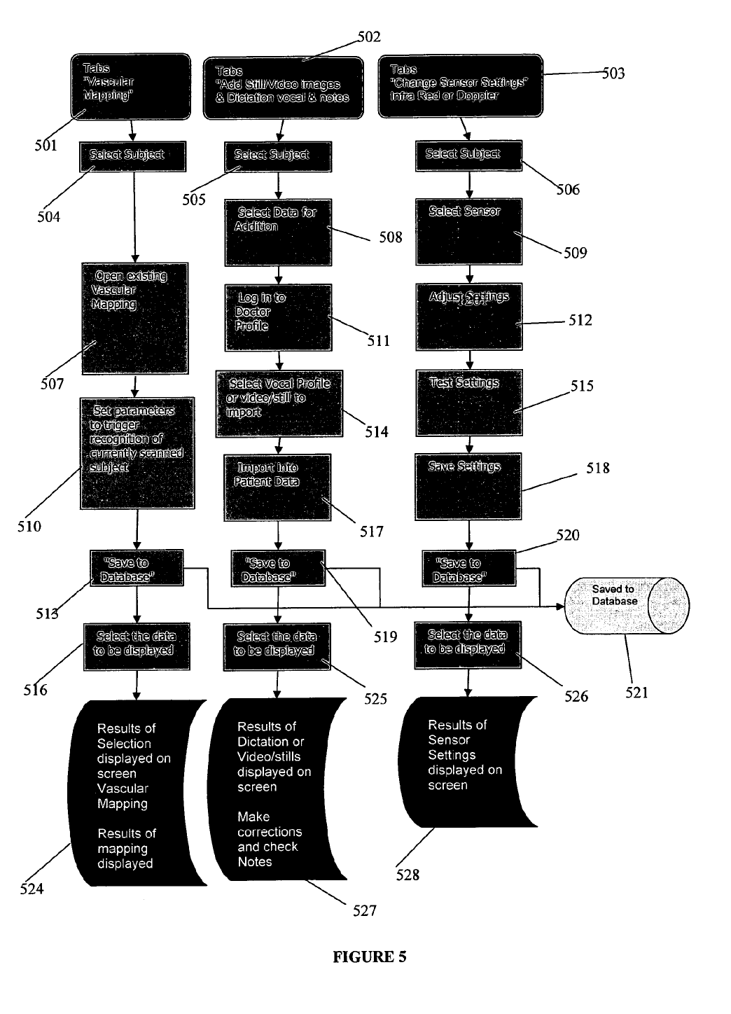

Once the user has set up their account they are then presented with a number of tabs 406 relating to different system functions. The flow charts illustrated in FIGS. 5 to 11 illustrate functionalities provided under various tabs which may be provided in one or more preferred embodiments of the present invention.

FIG. 5 in this instance depicts a number of tabs including vascular mapping 501, add still/video images and dictation vocal & notes 502 and change sensor settings (infrared or Doppler) 503. On selection of the vascular mapping tab 501 the user required to select a subject/patient 504 this may be done by entering the subjects name and other personal details or by entering the patient's reference number either manually or for example via scanning a barcode or the like associated with a patient's file. Once the system is provided with the relevant subject's details it then proceeds to open the vascular mapping application 507. The system the requests the user set a number of parameters to trigger recognition of the subject to be scanned 510, these specific parameters for the selected subject/patient are then stored 513 to the systems database 521. Once this is complete the user is then able to select the desired data for the given subject to be displayed 516, the selected data is then displayed on screen before vascular mapping is performed on the selected data and the results of the mapping displayed 524.

As with the selection of the vascular mapping tab 501, selection of the add still/video images and dictation vocal & notes tab 502 causes the system to prompt the user to select the desired subject/patient 505. Again this could involve entry of personal details pertaining to the subject or entering the patient's reference number etc. Once the user has selected the desired subject the system prompts the user to select the desired data form the patient records to which the addition is to be made 508. On selection of the desired data the user is then required to log into the relevant doctor's profile 511 to retrieve the relevant data such as vocal profiles (i.e. dictation files) and/or video and still images 514 to be appended to the selected patient data. The relevant vocal profiles and/or video and still images are then imported into the selected patient data 517 before being stored 519 to the systems database 521. The user may then select the newly annotated patient data 525 for review on screen to double check notes and make any corrections if necessary 527.

As in the cases of the above described tabs selection of the change sensor settings tab again causes the system to request selection of the desired subject/patient 506 for which the relevant senor settings are to be altered. Once the patient has been identified the system then prompts the user to select the senor for adjustment 509 i.e. the infrared (IR) senor or ultrasound/acoustic sensor. The user having selected the relevant sensor is then able to adjust the settings associated with the selected senor 512. After the settings for the given senor are adjusted the system proceeds to test the new setting 515 prior to saving the settings and the test results against the selected patient's profile. The new settings are then saved 520 to the systems database 521. The user may then elect to review the test results 526 of the new senor settings on screen 528 to determine if further adjustment is required.

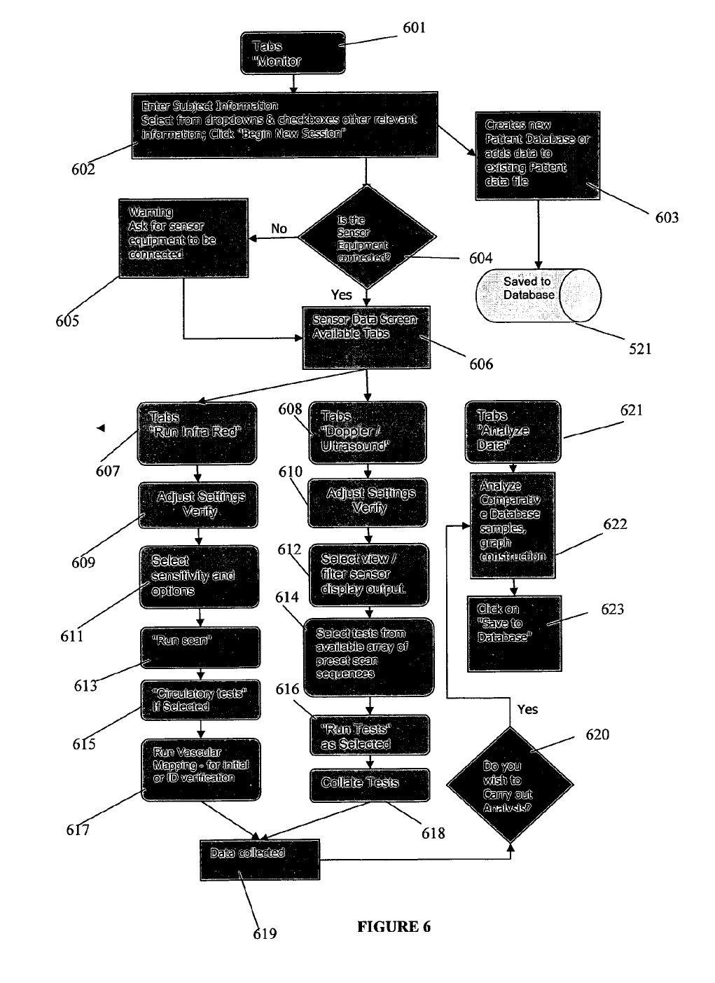

FIG. 6 depicts one possible process for data acquisition and analysis under a tab based system according to one embodiment of the present invention. In the depicted example the data acquisition process is initiated via selection of the monitor tab 601 on selection of the monitor tab the system request the user/operator to enter the relevant information relating to the subject 602. A set of dropdown menus and checkboxes etc could be provided to assist with entry of this information. The information from step 602 is then used to create a new patient data file or to appended data to an existing patient file as the case maybe 603, which is then stored to the systems database 521. Once the relevant information on the patient/subject has been captured, the user is able to start a new data collection session by clicking the begin new session button. On commencement of a new session the system firstly determines whether the senor equipment (i.e. apparatus 10) is connected 604. In the event that the sensor equipment is not connected the system advises the user that the senor equipment is not connected 605.

If the sensor equipment is connected (or connected subsequently as a result of step 605) the system then makes available a sensor data screen 606 which contains a number of tabs relating to the operation of the sensing equipment. In this example the sensor data screen includes two tabs one for actuating the infrared portion of the apparatus 10, run infrared tab 607, and one for actuating the ultrasonic portion of the apparatus 10, run Doppler/ultrasound tab 608. Selection of the run infrared tab 607 initiates data collection utilising the infrared portion of the apparatus 10. The user is then able to adjust/verify the current settings 609 for the IR portion of the apparatus i.e. intensity, wavelength etc of IR source. The user may then adjust the sensitivity of IR sensor in the IR portion of the apparatus in addition to selecting other options such as the selection of supplemental tests to be performed in addition to the main scan (e.g. various circulatory tests etc) 611.

As will be appreciated by those of skill in the art the adjustment of the various settings associated with the IR portion of the apparatus is required due to variation in patient skin type which results in a different IR absorption profile from patient to patient. Once the IR portion of the apparatus has been adjusted to suit the particular subject, scanning of a region of interest commences 613. On completion of the scan the system may then perform additional test such as circulatory tests etc 615 if selected under step 611 before running the vascular mapping application 617.

Data collection utilising the ultrasonic portion of the apparatus 10 is initiated by selection of the Doppler/ultrasound tab 608. As in the case of the selection of the run infrared tab 607 the selection of the Doppler/ultrasound tab 608 causes the system to prompts the user to adjust and verify the setting 610 associated with the ultrasonic portion of the apparatus 10 and more specifically the ultrasonic transducer. Once the settings have been verified the user is then free to select one or more views from and/or filter the output of the acoustic sensor 612. The user may then select a series of tests to perform from an array of preset scan sequences 614. Once the desired test sequence is selected the scan commences 616 and the results collated 618.

The system may then query 620 the user as to whether the wish to analyse the data collected 619 from both the IR and ultrasonic portions of the apparatus 10. In the event that analysis is not required at this particular juncture the collected data 619 my then be stored within the patient's data file for further processing via selection of the analyse data tab 621 i.e. once the current data acquisition session is complete. Selection of the analyse data tab 621 causes the system to compare the data collected at 619 against prior samples for the patient stored in their profile on the database and display the results as a graph construction 622 the resultant construction is then saved 623 to the database 521. As shown if the user's answer to query 620 is in the affirmative the system proceeds immediately to the comparative analysis of step 622 (i.e. comparison may occur in real-time as additional scans of the patient are performed).

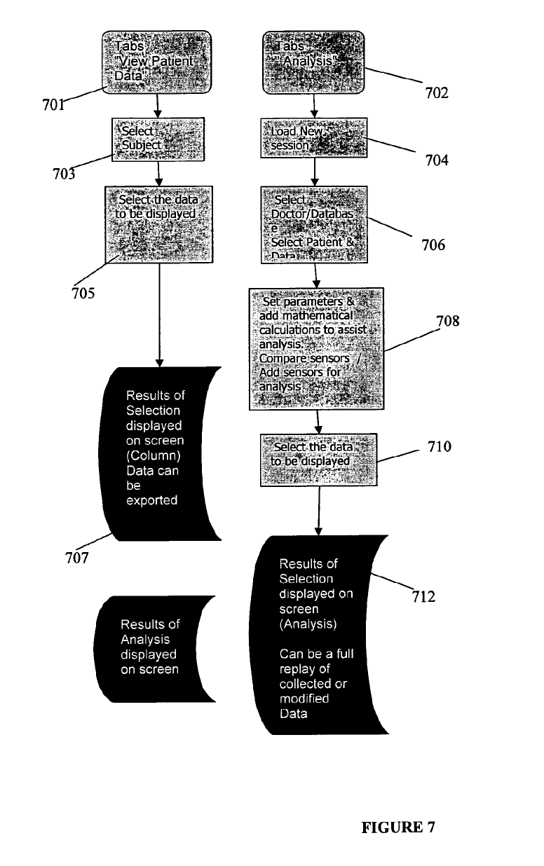

FIG. 7 depicts the operation of the view patient data 701 and Analysis 702 tabs according to one embodiment of the present invention. As shown selection of the view patient data tab 701 cause the system to requests the user to select the desired subject/patient 703. As with the above discussed examples this could be conducted in a variety of manners such as entry of personal details pertaining to the patient or patient ID number etc. The user is then required to select the desired data record pertaining to the selected subject for subsequent display 705. The selected information is then displayed 706, at which time the user may then be presented with the option to export the selected data for further analysis.

Selection of the analysis 702 tab in this instance facilitates the commencement of a new data session 704 which requires the user to select the desired patient data and relevant doctor's database (i.e. recent or past data annotated by consulting specialist or patient GP) 706. Once the relevant data for analysis is specified the system then allows the user to set a number parameters associated with the analysis to be performed as well as adding various mathematical calculations (eg statistical modelling etc) to further assist in the analysis of the specified data 708. At this stage the data on the senor/s utilised e.g. configuration etc may also be utilised as part of the analysis. The user can then select the portions of the analysed data 710 for subsequent display 710 for example the displayed data may simply be a full replay of collected (raw data) or modified data (i.e. post analysis).

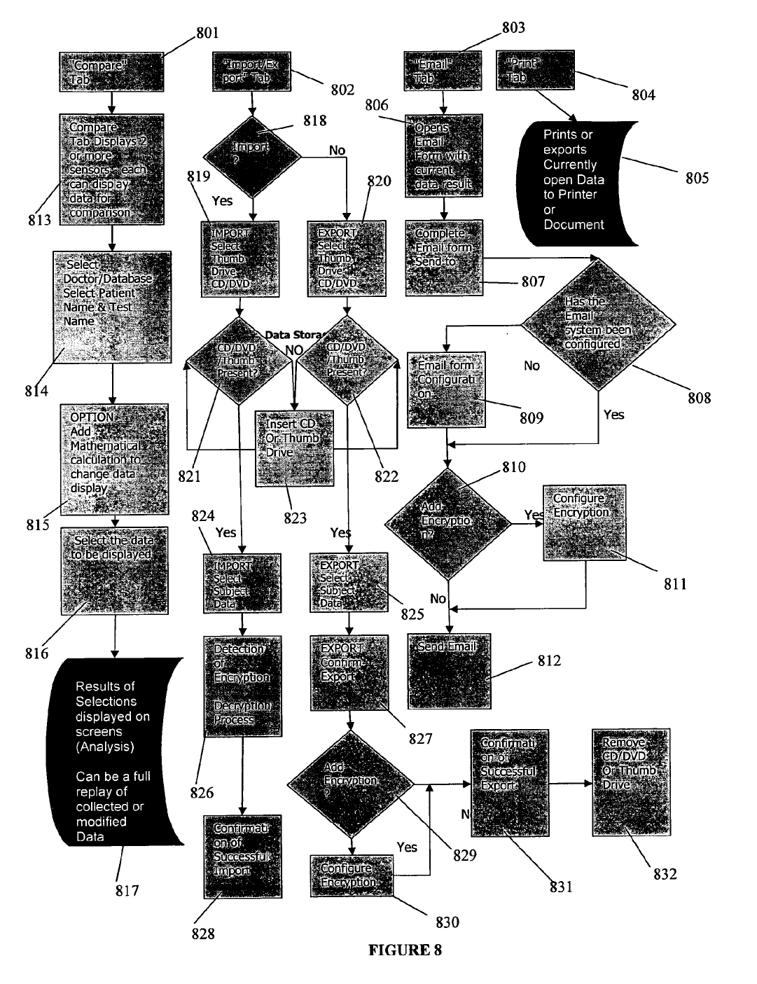

With reference to FIG. 8 there are illustrated a number of additional tabs which may be implemented under the system of the present invention. In this case the additional tabs include a compare tab 801, an import/export tab 802, an email tab 803 and a print tab 804.

As suggested the print tab 804 in this instance is provided to enable the user to print 805 the data they are currently viewing to a printer or to a file (e.g. a PDF file or the like). Likewise the email tab 803 is provided to allow the user to attach or send the data to a third party via email. Selecting the email tab causes the system to open an email form 806 with the current data results attached. On completion of the form 807 by the sender/user the system verifies if an email client has been configured 808, if so the system then queries the user as to whether the wish to encrypt the email 810. If encryption is desired the system proceeds to apply appropriate encryption 811 to the email before it is sent 812. In the event that the email client is not configure the system proceeds to configure the email client 809, prior to prompting the user for confirmation on whether they wish to use encryption 810 before sending the email 812. If no encryption in either case is required then the system proceeds to send the email 812.

The comparison tab 801 in this particular example provides the user with the ability compare data from two or more sensors. On selection of the compare tab 801 the user is provided with displays from two or more sensors 813 each display can display data for comparison. For each display the user is free to select desired patient data and relevant doctor's database (i.e. recent or past data annotated by consulting specialist or patient's GP) for comparison 814. The user is also able at this stage to add information as to the type of test being performed e.g. test name etc. The user may then be provided with the option to add additional analytical tools to modify the data display such as mathematical calculations etc 815. The user is then able to select various portions of the data displayed in each of the displays 816 (i.e. isolating a portion of the images within each display for display further detail etc), the resultant selections are then displayed on screen 817.

The import/export tab 802 enables the user in this instance to import data from backups etc or export the data to a select storage media etc. On selection of the import/export tab 802 the system queries the user as to the type of operation to be performed i.e. import/export 818. In the illustrated example the system simply queries the user as to whether an import is to be performed if the query is answered in the negative the system assumes that an export is to be performed in which case it request the user to specify the destination media 820 to which the data is to be exported e.g. thumb or CD/DVD drive etc. On receipt of the specified destination the system then checks to see if the destination is valid 822 i.e. thumb drive is mounted, blank CD/DVD inserted into drive etc. If the specified destination is not valid the system requests the user to respecify the desired destination 823. Once the system has determined that the specified destination for the export is valid it proceeds to export the selected data to the destination 825. On completion of this operation the system then confirms the export 827. The system then enables the user to encrypt the data 829, if encryption is required the system applies the appropriate level of encryption 830 before confirming completion of the export 831 whereon the system advises the user that it is safe to remove/unmount the relevant destination media.

In the case of a data import (i.e. the user answers in the affirmative at step 818), the system request the user to specify the drive, directory etc in which the data to be loaded is located 819. The system then verifies whether the specified drive or directory is valid 821 and if the specified import location is not valid the system requests the user to ensure that the specified drive is mounted etc or whether the directory is correctly specified 823. Once the system has determined that the specified drive or directory containing the data is valid it proceeds to import the specified data 824. The system the checks to see if the imported data has been encrypted and if so it proceeds to decrypt the data 826 before notifying the user of the successful importation of the specified data 828.

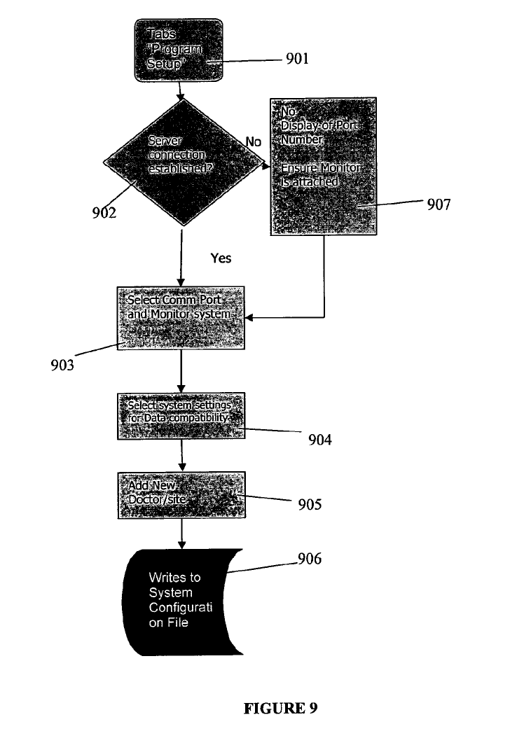

FIG. 9 depicts the operation of a set up tab 901 according to one embodiment of the present invention. As shown selection of the set up tab 901 causes the system to determine if a connection to a server is present 902 if so the system then asks the user to select the comm port and monitor system 903 (i.e. the port to which apparatus 10 is connected and other operating parameters of the apparatus). Once the comm and monitor settings are established the system then enables the user to adjust the system's data settings to ensure data compatibility 904. The option to link the system to additional external data sources such as doctor's databases and websites etc, may then be presented to the user 905 before the desired settings are written to the system config file 906. As illustrated in the event that a server connection is not present at step 902 the system proceeds to check the current port number assigned and ensure that a monitor is system is attached 907 before requesting the user to manually assign the require settings.

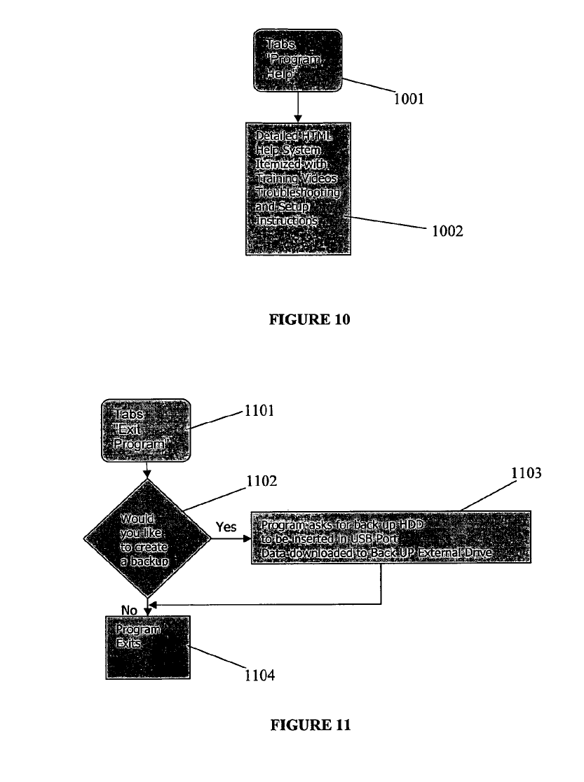

FIG. 10 illustrates the functionality of the help tab according to one embodiment of the present invention. As shown selection of the help tab 1001 pulls up a detail html based help system. 1002 which may be itemised with help videos, troubleshooting tips and setup instructions which the user is free to browse.

The operation of the exit tab 1101 is shown in FIG. 11. To exit the system the user selects the exit tab 1101 which causes the system to prompt the user to create a backup 1102. If the user elects to create a backup before exiting the system request the user specify a destination (e.g. an external hard drive or removable media g. a flash or USB drive or optical storage media such as a DVD or directory) to which the backup is to be stored 1103. Once the backup is created the system shuts down 1104. In the event that the user does not wish to perform a backup at step 1103 the system proceeds to shutdown 1104.

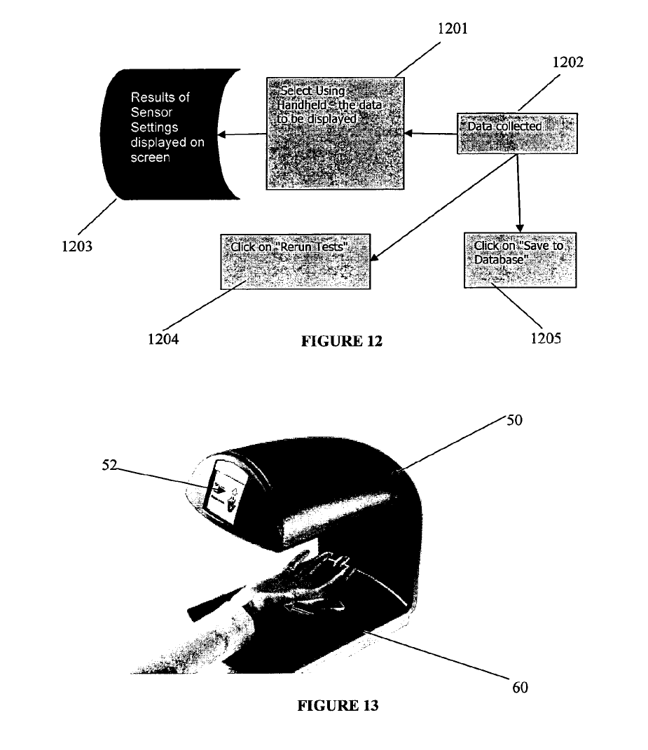

Illustrated in FIG. 12 is a flow chart relating to the display and collection data on via a handheld device 1201 such as a PDA, Smartphone or the like. As shown the data collected 1202 for example via the process discussed in relation to FIG. 6 may be sent to the handheld device 1201. The user then has the option to select data for display on screen 1203 or to rerun the test 1204. Any data obtained during rescanning on selection of the rerun test option 1204 may then be saved to the system database 1205.

The combined vein and artery information obtained utilising the infrared and ultrasound portions of the apparatus 10 have various uses including, for example, the locating of objects, such as needles, in a vessel (as discussed) and also for biometric identification. The unique layout of veins and arteries in a portion of a body of a user may then be utilised for identification purposes. In a biometric identification embodiment, preferably the apparatus is a fixed apparatus adapted to allow location of a portion of a user's body, such as their hand, adjacently. FIG. 13 illustrates an embodiment of the invention directed towards biometric identification. The apparatus 50 rests on a surface (or, alternatively, may be mounted on, or within, a wall) and a user places a portion of their body 60, in a preferred embodiment their hand, within the apparatus 50. The apparatus 50 then determines the location of the vessels of the portion of the body. A display 52 is provided on the apparatus which may display the vessel location information and/or other pertinent information such as instructions, whether the user was authenticated, and/or security details. Once scanned, the vein and artery layout as determined by the infrared and ultrasound portions may then be detected and either stored for later comparison, or compared to a stored layout. If the detected layout is the same as a stored layout (or at least within predetermined tolerances) then authentication of the user via vessel biometric identification may be achieved. Identification of a user may further require an additional step such as, for example, the scanning of an identity card or the inputting of a digital number combination (e.g. personal pin number). Hygienic sanitisation of the portion of the body (e.g. their hands) may also be performed electronically by infrared or other suitable means.

It will be appreciated by those of skill in the art that while the biometric identification device of FIG. 13 requires the subject to insert a portion of their body such as their hand into or under a portion of the apparatus other constructions of the device are possible. For example the device could be a hand held device which is scanned over the relevant portion of the subject to verify their identification. Alternatively the subject could be required to place the relevant portion of their body onto a plate/screen behind which the infrared and ultrasound portions apparatus may be housed.

It is to be understood that the above embodiments have been provided only by way of exemplification of this invention, and that further modifications and improvements thereto, as would be apparent to persons skilled in the relevant art, are deemed to fall within the broad scope and ambit of the present invention described herein.

1. An apparatus for non-invasively locating blood vessels within a portion of a body, the apparatus comprising:
a portion utilising infrared to locate at least some of the vessels, and
a portion utilising ultrasound to locate at least some of the vessels.

2. The apparatus of claim 1 wherein the portion utilising infrared comprises at least one infrared source for directing infrared radiation toward said body portion.

3. The apparatus of claim 2 further comprising at least one infrared sensor for receiving infrared radiation reflected from said body portion.

4. The apparatus of claim 1 wherein the portion utilising ultrasound comprises at least one ultrasonic transducer for directing ultrasonic energy toward said body portion.

5. The apparatus of claim 4 further comprising at least one receiver for receiving reflected ultrasonic energy from said body portion.

6. The apparatus of claim 5 wherein the ultrasonic energy is pulsed.

7. The apparatus of claim 1 wherein the portion utilising infrared primarily locates venous vessels and the portion utilising ultrasound primarily locates arterial vessels.

8. The apparatus of claim 7 wherein the portion utilising infrared primarily locates the venous vessels based on the absorptive spectra of said body portion.

9. The apparatus of claim 7 wherein the portion utilising ultrasound locates arterial vessels by measuring blood flow through the vessels within said body portion.

10. The apparatus of claim 9 wherein directionality of the blood flow through the vessels is determined utilising Doppler shift detection.

11. The apparatus of claim 9 wherein the apparatus is further configured to use range gating to determine blood velocity relative to each vessel's cross section.

12. The apparatus of claim 1 further comprising a display for producing images of the vessels within said body portion.

13. The apparatus of claim 12 wherein the display is remote from the apparatus.

14. The apparatus of claim 13 wherein the display is hand held touch screen display.

15. The apparatus of claim 12 wherein the venous vessels and arterial vessels are displayed simultaneously in differing colours.

16. The apparatus of claim 1 further comprising an output for the transfer of data relating to the location of the vessels captured by the apparatus for further processing.

17. The apparatus of claim 16 wherein the output comprises a transmitter for wireless transmission of the data.

18. An apparatus for non-invasively locating vessels within a region of interest within a body, the apparatus comprising:
at least one infrared source for directing infrared radiation toward the region of interest;
at least one infrared sensor for receiving infrared radiation reflected from said region of interest;
at least one ultrasonic transducer for directing ultrasonic energy toward the region of interest;
at least one acoustic senor for receiving ultrasonic energy reflected from said region of interest; and
wherein the apparatus is adapted to locate a first vessel type within the area of interest based on the reflected infrared radiation and to locate a second vessel type within the area of interest based on the reflected ultrasonic energy.

19. The apparatus of claim 18 wherein the ultrasonic energy is pulsed.

20. The apparatus of claim 18 wherein the ultrasonic energy is emitted as a plurality of beams directed a differing angles to the area of interest.

21. The apparatus of claim 18 wherein the first vessel type is located based on the absorptive spectra of the region of interest.

22. The apparatus of claim 18 wherein the second vessel type is located by measuring fluid flow through the vessel.

23. The apparatus of claim 22 wherein directionality of the fluid flow through the vessels is determined utilising Doppler shift detection.

24. The apparatus of claim 22 wherein the apparatus is further configured to use range gating to determine fluid velocity relative to each vessel's cross section.

25. The apparatus of claim 18 wherein the first vessel type are venous vessels and the second vessel type are arterial vessels.

26. The apparatus of claim 18 further comprises a display for producing images of the vessels within said region of interest.

27. The apparatus of claim 26 wherein the display is remote from the apparatus.

28. The apparatus of claim 27 wherein the display is hand held touch screen display

29. The apparatus of claim 26 wherein the first and second vessel types are displayed simultaneously in differing colours.

30. The apparatus of claim 18 further comprising an output for the transfer of data relating to the location of the vessels captured by the apparatus for further processing.

31. The apparatus of claim 30 wherein the output comprises a transmitter for wireless transmission of the data.

32.-39. (canceled)

40. A system for non-invasively locating vessels within a region of interest within a body, said system comprising:
an apparatus, said apparatus including:
at least one infrared source for directing infrared radiation toward the region of interest;
at least one infrared sensor for receiving infrared radiation reflected from said region of interest;
at least one ultrasonic transducer for directing ultrasonic energy toward the region of interest;
at least one acoustic senor for receiving ultrasonic energy reflected from said region of interest;
at least one processor coupled to said apparatus wherein said at least one processor is adapted to:
determine the locations of vessels of a first vessel type within the area of interest based on the reflected infrared radiation; and
determine the locations of vessels of a second vessel type within the area of interest based on the on the reflected ultrasonic energy.

41. The system of claim 40 wherein the ultrasonic energy is pulsed.

42. The system of claim 40 wherein the ultrasonic energy is emitted as a plurality of beams directed a differing angles to the area of interest.

43. The system of claim 40 wherein determination of the locations of vessels of the first vessel type includes identifying and mapping one or more regions within the area of interest having the highest rate of abortion of the incident infrared radiation.

44. The system of claim 40 wherein determination of the locations of vessels of the second vessel type includes determining the direction and/or the rate of fluid flow through the vessels.

45. The system of claim 44 wherein directionality of the fluid flow through the vessels is determined utilising Doppler shift detection.

46. The system of claim 44 wherein the rate of fluid flow through the vessels is determined utilising range gating to determine fluid velocity relative to each vessel's cross section.

47. The system of claim 40 wherein the first vessel type are venous vessels and the second vessel type are arterial vessels.

48. The system of claim 40 further comprises a display for producing images of the vessels within said region of interest.

49. The system of claim 48 wherein the display is remote from the apparatus.

50. The system of claim 49 wherein the display is hand held touch screen display

51. The system of claim 47 wherein the first and second vessel types are displayed simultaneously in differing colours.

52. The system of claim 40 further comprising an output for the transfer of data relating to the location of the vessels captured by the apparatus for further processing.

53. The system of claim 52 wherein the output comprises a transmitter for wireless transmission of the data.

54. The system of claim 40 wherein the at least one processor is adapted to map the positions of the first and second vessel types within the area of interest.

55. The apparatus of claim 18 wherein the apparatus is configured to map the positions of the first and second vessel types within the area of interest.
